(19) United States
(12) Patent Application Publication
(10) Pub. No.: US 2007/0100449 A1
(43) Pub. Date: May 3, 2007

(54) Injectable soft tissue fixation technique

(76) Inventors: Michael O'Neil, W. Barnstable, MA (US); Alonzo Cook, Lakeville, MA (US); John Voellmicke, Cumberland, RI (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/262,904

(22) Filed: Oct. 31, 2005

(51) Int. Cl. A61F 2/08; A61B 17/70

(52) U.S. Cl. 623/13.14; 606/61

(57) ABSTRACT

The present invention is directed toward a method for easily and securely attaching soft tissue graft materials to bone without puncturing the graft material and to provide for regeneration of bone removed for attachment.

### BACKGROUND OF THE INVENTION

1. Field of the Invention

This invention relates to soft tissue, matrixes and/or grafts that are affixed to bony tissue via intra-operatively dispensed materials that are preferably osteoinductive. More specifically the invention is directed to methods for easily and securely attaching soft tissue graft materials to bone without puncturing the graft material and to provide for regeneration of bone removed for attachment while providing little or no profile.

2. Related Art

Soft tissues fixation techniques can be segregated into puncturing and non-puncturing designs. The majority of designs are puncturing; this includes screws, pins, sutures, staples, etc. One patent, U.S. Pat. No. 5,681,310, specifically requires puncturing the flexible graft material with a plurality of fasteners to secure intervertebral devices.

Non-puncturing designs include staples that straddle the soft tissue. U.S. Pat. No. 5,209,756 utilizes a floating “stirrup” staple through which the soft tissue is wrapped and engages the tines of the staple to secure without puncturing the graft. Other non-puncturing designs disclosing wedging of soft tissue by bony dowels are described for knee ACL surgery.

Some non-puncturing art has been found to disclose the use of flowable and curable polymers.

U.S. Pat. No. 4,065,817 discloses a bone prosthesis with a tubular support member with lateral openings and cement injected through the tubular member to secure it in place.

U.S. Pat. No. 6,610,079 discloses a surgical implant with a sleeve to receive a flowable medium at one transverse opening.

US20030083662 discloses a preformed element (anchor or screw) with proximal and distal apertures that is positioned within a bone pilot hole and cavity. Injecting a hardenable material through apertures into the pilot hole and cavity secures the preformed element. Hardenable material can be a bone substitute.

US20040049194 discloses a soft tissue fixation method of piercing the soft tissue and deploying a material in a flowable state and changing the state to such that the material forms an interference fit and molding a portion of the material that is not in the opening to hold soft tissue against the bone.

### BRIEF DESCRIPTION OF THE DRAWINGS

FIGS. 1a-1b depict one embodiment of this invention wherein a soft tissue implant is shown secured into vertebral bodies.

FIG. 2 depicts another embodiment of this invention wherein the securement of the implant further comprises a coil.

FIG. 3 depicts yet another embodiment of this invention wherein the securement of the implant further comprises a shape memory securing device.

### SUMMARY OF THE INVENTION

The present invention relates to a method for securing a soft tissue implant into bone comprising the steps of:

a) providing a hole in the bone;

b) inserting an end of the soft tissue implant into the hole; and

c) filling the hole with a curable material.

Among the advantages of this invention's soft tissue graft fixation techniques include no puncturing of the graft material that can lead to failure upon loading; replacement of bone removed to affix graft with an osteo-regenerative material allowing for bony regeneration and Sharpie's fibers integration of the soft tissue; and low or no profile fixation.

### DETAILED DESCRIPTION OF PREFERRED EMBODIMENTS OF THE INVENTION

This invention is directed to a method for easily and securely attaching soft tissue implants to bone without puncturing the implant and to provide for regeneration of bone removed for attachment while providing little or no profile. In preferred embodiments the implants are soft tissue, matrixes and/or grafts that are affixed to bony tissue via intra-operatively dispensed osteoinductive materials.

No prior art appears to disclose the use of hardenable or curable osteo-inductive material to facilitate fixation of allo or xeno-graft ECM's, including small intestine submucosa (“SIS”) in the manner hereinafter described. No art appears to disclose one piece soft tissue graft construction used with a hardenable injectable soft tissue fixation technique material to affix a flexible graft material to a bony substrate. Published art appears not to disclose the use of a blind hole (i.e., a hole with a closed bottom) to facilitate graft securement.

The soft tissue implants may comprise ligaments, tendons, and muscle. More specific examples for spinal applications include the anterior longitudinal ligament, the posterior longitudinal ligament, the interspinous ligament, the ligamentum flavum, and the supraspinous ligament. Additional implants may comprise regenerative membranes for guided tissue regeneration for periodontal ligament repair, for tendon repair such as the Achilles tendon, supraspinatus tendon for rotator cuff repair or anterior cruciate ligament repair.

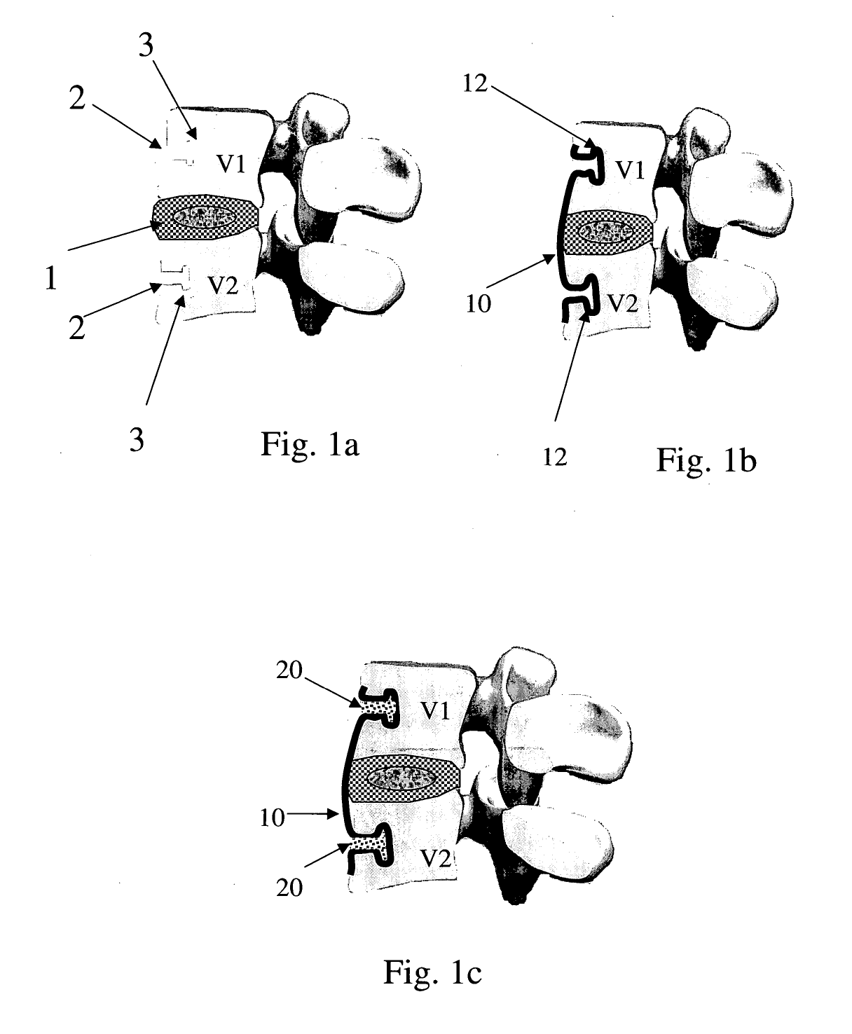

FIGS. 1a-1c depict one embodiment of this invention wherein the injectable soft tissue fixation technique is used to attach soft tissue to bone. Referring to FIG. 1a, holes 2 are made in adjacent vertebral bodies V1 and V2. Intervertebral object 1 generally may be a fusion cage, an artificial disc, or an intervertebral disc, dependent on the surgical procedure being performed. Also, the surgical procedure may simply be reattaching native tissue that has lost its bone anchoring through, for example, an traumatic event. Optional undercuts 3 are shown in V1 and V2 in the event additional anchoring capability for the spinal implant is desired. It should be noted that although this embodiment is directed to replacement of an anterior spinal ligament, which may arise when performing a fusion operation, an insertion of an artificial disc of repair of the intervertebral disc, or reattachment procedure of native tissue, the concepts of this invention of equally applicable to other procedures wherein soft tissue is desired to be attached to bone without puncturing the soft tissue. Referring to FIG. 1b soft tissue implant 10 is inserted into the bone holes, and may comprise endcaps 12. Endcaps 12 are optional and are designed to increase anchoring ability of implant 10. Also although FIG. 1b is depicted to fully line the bone holes, it would be appreciated by one skilled in the art that only a portion of the bone hole may need to be lined or otherwise inserted into the bone holes to an extent to provide sufficient holding ability to permit the implant to remain secure in the bone in conjunction with a curable material such as those hereinafter disclosed.

FIG. 1c represents a completed procedure wherein implant 10 has be secured into the holes with curable material 20. The term “curable material” is intended to describe injectable materials that are flowable and hardenable materials enabling eventually firm securement of the soft tissue implant.

As noted above, the soft tissue implant can be just simply placed into the hole directly or it can be preformed with endcaps in manufacturing or the operating room and subsequently placed into the hole. The soft tissue graft may include micro or macroscopic slots, ridges or other features to allow the injectable to flow into and through the soft tissue graft against the bone further enhancing securement.

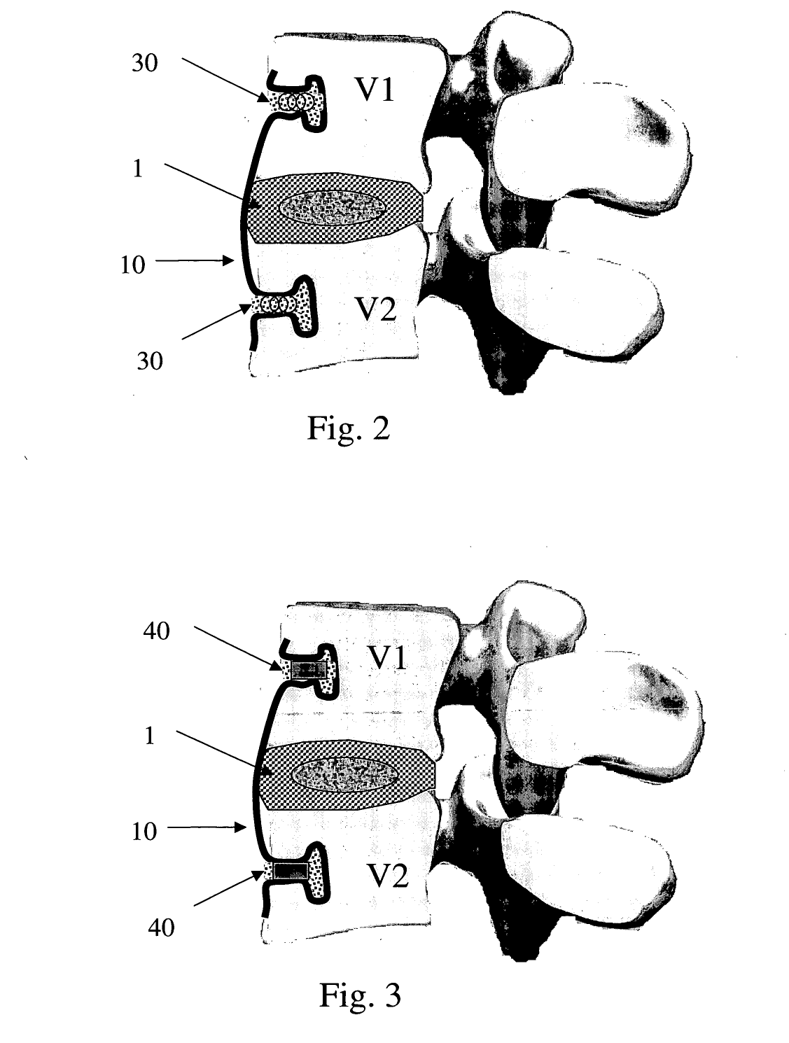

Alternatively, the soft tissue implant can be held in place with securing devices such as a metal (e.g., Nitinol) or plastic (e.g., polyurethane), preferably degradable polymer, spring or coil that provides the initial mechanical strength to position the soft tissue graft during injection of the curable material. FIG. 2 describes the aforementioned securing device coil 30 is embedded into the curable polymer. Preferably coil 30 is made from a material that is compatible with the polymer and with the bone that replaces the polymer. FIG. 3 depicts an alternate embodiment, wherein the securing device 40 is placed in the defect for initial support and can be compressible for insertion, but maintaining shape memory so that it returns to a size that secures the implant in the hole. The hole may then be filled with a fluid (e.g. polymer solution) that creates a cross-linking reaction that results in the hardening of the polymer. The liquid may be a degradable polymer that is subsequently replaced by bone.

Additionally a small balloon comprised of polymeric materials (polyethylene terephthalate, polyurethane, or nylon) about the size of a small marble (e.g., 15 mm diameter) can be inserted into a small pilot hole in the bone behind the soft tissue implant and then expanded under pressure with a curable material, such as polymethylmethacrylate, to locally compress the surrounding cancellous bone and create the undercut (i.e., a solid sphere beneath the surface of the bone). The material will cure inside the balloon and remain in the bone.

One skilled in the art would appreciate that other methods are applicable according to the objectives of this invention and that the order of steps according to any particular method are not limitative. Such non-limiting method examples include:

a method of comprising the steps of: (1) creating a pilot hole in bone, (2) creating a void in the bone with an expandable tool inserted into the pilot hole, (3) inserting an end of the tissue implant or native tissue into the void, and (4) filling the void with a curable material; or

a method comprising the steps of: (1) creating a pilot hole in bone, (2) inserting an end of a tissue implant or native tissue into the pilot hole, (3) expanding a small balloon filled with curable material inside the pilot hole and leaving the balloon inside the bone.

Examples of materials suitable for use as a soft tissue implant of this invention include but are not limited to biocompatible polymers. A variety of biocompatible polymers, both bioabsorbable and nonbioabsorbable, can be used as the implant according to the present invention. The biocompatible polymers can be synthetic polymers, natural polymers or combinations thereof. As used herein the term “synthetic polymer” refers to polymers that are not found in nature, even if the polymers are made from naturally occurring biomaterials. The term “natural polymer” refers to polymers that are naturally occurring.

In embodiments where the implants includes at least one synthetic polymer, suitable biocompatible synthetic polymers can include polymers selected from the group consisting of aliphatic polyesters, poly(amino acids), copoly(ether-esters), polyalkylenes oxalates, polyamides, tyrosine derived polycarbonates, poly(iminocarbonates), polyorthoesters, polyoxaesters, polyamidoesters, polyoxaesters containing amine groups, poly(anhydrides), polyphosphazenes, poly(propylene fumarate), polyurethane, poly(ester urethane), poly(ether urethane), and blends and copolymers thereof.

Of the foregoing, useful non-bioabsorbable polymers include, but are not limited to polyacrylates, ethylene-vinyl acetates (and other acyl-substituted cellulose acetates), polyester (Dacron®), poly(ethylene terephthalate), polypropylene, polyethylene, polyurethanes, polystyrenes, polyvinyl oxides, polyvinyl fluorides, poly(vinyl imidazoles), chlorosulphonated polyolefins, polyethylene oxides, polyvinyl alcohols (PVA), polytetrafluoroethylenes, nylons, and combinations thereof.

Suitable synthetic polymers for use in the present invention can also include biosynthetic polymers based on sequences found in collagen, laminin, glycosaminoglycans, elastin, thrombin, fibronectin, starches, poly(amino acid), gelatin, alginate, pectin, fibrin, oxidized cellulose, chitin, chitosan, tropoelastin, hyaluronic acid, silk, ribonucleic acids, deoxyribonucleic acids, polypeptides, proteins, polysaccharides, polynucleotides and combinations thereof.

For the purpose of this invention aliphatic polyesters include, but are not limited to, homopolymers and copolymers of lactide (which includes lactic acid, D,L- and meso lactide); glycolide (including glycolic acid); ε-caprolactone; p-dioxanone (1,4-dioxan-2-one); trimethylene carbonate (1,3-dioxan-2-one); alkyl derivatives of trimethylene carbonate; δ-valerolactone; β-butyrolactone; γ-butyrolactone; ε-decalactone; hydroxybutyrate; hydroxyvalerate; 1,4-dioxepan-2-one (including its dimer 1,5,8,12-tetraoxacyclotetradecane-7,14-dione); 1,5-dioxepan-2-one; 6,6-dimethyl-1,4-dioxan-2-one; 2,5-diketomorpholine; pivalolactone; α,α diethylpropiolactone; ethylene carbonate; ethylene oxalate; 3-methyl-1,4-dioxane-2,5-dione; 3,3-diethyl-1,4-dioxan-2,5-dione; 6,6-dimethyl-dioxepan-2-one; 6,8-dioxabicycloctane-7-one and polymer blends thereof. Aliphatic polyesters used in the present invention can be homopolymers or copolymers (random, block, segmented, tapered blocks, graft, triblock, etc.) having a linear, branched or star structure. Other useful polymers include polyphosphazenes, co-, ter- and higher order mixed monomer based polymers made from L-lactide, D,L-lactide, lactic acid, glycolide, glycolic acid, para-dioxanone, trimethylene carbonate and ε-caprolactone.

In one embodiment, the implant includes at least one natural polymer. Suitable examples of natural polymers include, but are not limited to, fibrin-based materials, collagen-based materials, hyaluronic acid-based materials, glycoprotein-based materials, cellulose-based materials, silks and combinations thereof.

In yet another embodiment, the implant includes a naturally occurring extracellular matrix material (“ECM”), such as that found in the stomach, bladder, alimentary, respiratory, urinary, integumentary, genital tracts, or liver basement membrane of animals. Preferably, the ECM is derived from the alimentary tract of mammals, such as cows, sheep, dogs, cats, and most preferably from the intestinal tract of pigs. The ECM is preferably small intestine submucosa (“SIS”), which can include the tunica submucosa, along with basilar portions of the tunica mucosa, particularly the lamina muscularis mucosa and the stratum compactum.

For the purposes of this invention, it is within the definition of a naturally occurring ECM to clean and/or comminute the ECM, or to cross-link the collagen within the ECM. It is also within the definition of naturally occurring extracellular matrix to fully or partially remove one or more components or subcomponents of the naturally occurring matrix. However, it is not within the definition of a naturally occurring ECM to extract, separate and purify the natural components or sub-components and reform a matrix material from purified natural components or sub-components. Also, while reference is made to SIS, it is understood that other naturally occurring ECMs (e.g., stomach, bladder, alimentary, respiratory or genital submucosa, and liver basement membrane), whatever the source (e.g., bovine, porcine, ovine) are within the scope of this invention. Thus, in this application, the terms “naturally occurring extracellular matrix” or “naturally occurring ECM” are intended to refer to extracellular matrix material that has been cleaned, disinfected, sterilized, and optionally cross-linked.

In other embodiments of the present invention, the implant can be formed from elastomeric copolymers such as, for example, polymers having an inherent viscosity in the range of about 1.2 dL/g to 4 dL/g, more preferably about 1.2 dL/g to 2 dL/g, and most preferably about 1.4 dL/g to 2 dL/g as determined at 25° C. in a 0.1 gram per deciliter (g/dL) solution of polymer in hexafluoroisopropanol (HFIP). Suitable elastomers also preferably exhibit a high percent elongation and a low modulus, while possessing good tensile strength and good recovery characteristics. In the preferred embodiments of this invention, the elastomer exhibits a percent elongation greater than about 200 percent and preferably greater than about 500 percent. In addition to these elongation and modulus properties, the elastomers should also have a tensile strength greater than about 500 psi, preferably greater than about 1,000 psi, and a tear strength of greater than about 50 lbs/inch, preferably greater than about 80 lbs/inch.

Exemplary biocompatible elastomers are selected form the group consisting of ε-caprolactone, glycolide, lactide, p-dioxanone, trimethylene carbonate and combinations thereof and include, but are not limited to, elastomeric copolymers of ε-caprolactone and glycolide with a mole ratio of ε-caprolactone to glycolide of from about 35:65 to about 65:35, more preferably from 45:55 to 35:65; elastomeric copolymers of ε-caprolactone and lactide (including L-lactide, D-lactide, blends thereof, and lactic acid polymers and copolymers) where the mole ratio of ε-caprolactone to lactide is from about 95:5 to about 30:70 and more preferably from 45:55 to 30:70 or from about 95:5 to about 85:15; elastomeric copolymers of p-dioxanone (1,4-dioxan-2-one) and lactide (including L-lactide, D-lactide, blends thereof, and lactic acid polymers and copolymers) where the mole ratio of p-dioxanone to lactide is from about 40:60 to about 60:40; elastomeric copolymers of ε-caprolactone and p-dioxanone where the mole ratio of ε-caprolactone to p-dioxanone is from about from 30:70 to about 70:30; elastomeric copolymers of p-dioxanone and trimethylene carbonate where the mole ratio of p-dioxanone to trimethylene carbonate is from about 30:70 to about 70:30; elastomeric copolymers of trimethylene carbonate and glycolide (including polyglycolic acid) where the mole ratio of trimethylene carbonate to glycolide is from about 30:70 to about 70:30; elastomeric copolymers of trimethylene carbonate and lactide (including L-lactide, D-lactide, blends thereof, and lactic acid polymers and copolymers) where the mole ratio of trimethylene carbonate to lactide is from about 30:70 to about 70:30; and blends thereof. Other examples of suitable biocompatible elastomers are described in U.S. Pat. No. 5,468,253.

The curable materials include materials that are flowable and hardenable (e.g., that change state, undergo a phase transition, or harden, based on any process, e.g., chemical, irradiation, phase transition, etc.) General examples of these materials include current bone cements, resorbable or non-resorbable polymers, tissue adhesives, biological adhesives, or curable polymers that rigidize or harden upon irradiation (such as infrared radiation), exposure to heat energy or any other suitable energy source compatible with the curing process of the flowable material.

More specific examples include PMMA bone cements including those made from methyl acrylic and polymethyl acrylic, or methyl methacrylic styrene copolymers with or without the addition of barium sulphate.

Additionally the curable material may be a two or more component polymer which cures once the two components have been mixed after an elapsed time or cures by irradiation, or cures with any type of applied energy or cures by body heat.

Examples of suitable curable materials which can be used in the invention, include, without limitation, such materials as polypropylene fumarate, polymethyl methacrylate (PMMA), and various cross linking polymers. These are merely examples which can be used in a two or more component system. Other materials can be used.

Other examples of materials which can be used include, without limitation, a methacrylate copolymer which undergoes a phase transition when exposed to heat. There are other materials that could be employed, including materials that flow upon cooling and harden with an increase in temperature, for example, a protein based polymer.

Additionally, the components of the curable material can be powders or a liquid and a powder or combination of liquids and powders. Further, more than two components can be used, for example, three or more components.

For example, if one of the components is a powder and the other a liquid, the mixing device mixes the powder with the liquid to cause the initiation of the polymerization of the mixed polymer. An advantage of using a liquid/powder system is that the two components, one being liquid and one being powder, have longer or indefinite shelf lives as compared with two liquids. Certain two component polymers which are both liquids have a shelf life so that the liquids may start to gel or polymerize by themselves prior to mixing. Preferably, the fluid is polyvinylpyrrolidone (PVP) which initiates the cross-linking and the powder preferably is polypropylene fumarate (PPF). Alternatively, the two components may comprise PMMA or some other two or more component system such as calcium phosphate saline solution. Further, both components may be flowable particulates.

Other suitable curable materials include those said to have structural properties appropriate for load-bearing orthopaedic implants. For example, U.S. Pat. No. 5,990,194 to Dunn et. al. discloses biodegradable thermoplastic and thermosetting polymers for use in providing syringeable, in-situ forming, solid biodegradable implants.

U.S. Pat. No. 6,264,659 to Ross et. al. describes a thermoplastic implant material that is heated to a predetermined high temperature for injection from a needle. After injection, the thermoplastic material is cooled by the body temperature for setting of the thermoplastic material to a non-flowing state. The preferred thermoplastc material is said to be gutta-percha or gutta-percha compound.

Other curable materials include synthetic bone substitutes. For example, resorbable and injectable calcium phosphates, such as the material offered by Synthes-Stratec, Inc. under the Norian Skeletal Repair System® brand name. An example of a non-resorbable bone substitute is an injectable terpolymer resin with combeite glass-ceramic reinforcing particles, such as the material offered by Orthovita, Inc. under the Cortoss® brand name. Cortoss® is purported to have strength comparable to human cortical bone.

Additionally the curable material may include a resorbable polymer, e.g., polycaprolactone (PCL), which will slowly resorb during the natural healing process. The polymer may also include a non-resorbable polymer, e.g., polypropylene, polyacetal, polyethylene or polyurethane. The polymer may also include a blend of different resorbable polymers that resorb at different rates, e.g., blends of two or more of the following polymers: polycaprolactone (PCL), poly-1-lactic acid, poly-DL-lactic acid, polyglycolic acid, polydioxanone, polyglyconate, polytrimethylene carbonate, and copolymers of poly-L-lactic acid, poly-DL-lactic acid, polyglycolic acid, polydioxanone, polyglyconate, polytrimethylene carbonate, poly(hydroxyalkonates) (PHB, PHO, PHV), polyorthoesters, polyanhydrides, poly(pseudo-amino acids), poly(cyanoacrylates), poly(ester-anhydrides), polyoxalates, and polysaccharides. Other suitable polymers include poly-4-hydroxybutyrate (4PHB) and poly(alkylene oxalates).

The use of crosslinking agents that are light curable can also be used. In preferred embodiments, the cross-linkable component is UV curable. Examples of UV curable cross-linkable components are disclosed in Biomaterials (2000), 21:2395-2404 and by Shastri in U.S. Pat. No. 5,837,752, the entire teachings of which are incorporated herein by reference.

In some embodiments, the curable material comprises a polymer and a cross-linking agent. In some embodiments, the curable material may further comprise a monomer. In some embodiments, the curable material may further comprise an initiator. In some embodiments, the curable material may further comprise an accelerant.

A preferred embodiment incorporates additives, fillers, and/or porosity that encourages bony ingrowth and allows for bony tissue regeneration. An example is the use of poly amino acids or poly anhydrides filled with tricalcium phosphate, calcium sulfate or hollow PMMA microspheres such as Bioplant HTR particles (available from Kerr Corporation, Orange Calif. 92867) The curable materials are designed to provide mechanical fixation and bony regeneration.

It will be apparent to those skilled in the art that numerous injection devices and supporting devices can be appropriate for delivery of the curable material(s). The simplest devices can be in the form of a syringe, or an injection device can be described as an application gun. Some curable materials may be comprised of two or more compounds mixed together to form an injectable material that hardens or cures in-situ through a chemical reaction. Mixing can occur in a separate device or an injection device can have a means for storing multiple compounds and mixing them during the injection process. For example, the manual injection device for Orthovita's Cortoss® includes dual cartridges wherein polymerization is initiated when Cortoss® is expressed through a “static mix-tip”.

Another aspect of the current invention is the ability of the injectable curable material to release growth factors (proteins) that enhance regeneration of the surrounding bone.

For example, rhGDF-5 combined with collagen particles or polyanhydrides can be injected and cured in situ. The growth factor is then slowly released and acts on surrounding cells to induce bone formation. Alternatively chemotactic agents may be delivered to promote cellular infiltration.

Other useful curable, injectable compositions comprise bioactive agents. “Bioactive agents,” as used herein, can include one or more of the following: chemotactic agents; various proteins (e.g., short term peptides, bone morphogenic proteins, collagen, hyaluronic acid, glycoproteins, and lipoprotein); cell attachment mediators; biologically active ligands; integrin binding sequence; ligands; various growth and/or differentiation agents and fragments thereof (e.g., epidermal growth factor (EGF), hepatocyte growth factor (HGF), vascular endothelial growth factors (VEGF), fibroblast growth factors (e.g., bFGF), platelet derived growth factors (PDGF), insulin derived growth factor (e.g., IGF-1, IGF-II) and transforming growth factors (e.g., TGF-β I-III), parathyroid hormone, parathyroid hormone related peptide, bone morphogenic proteins (e.g., BMP-2, BMP-4; BMP-6; BMP-7; BMP-12; BMP-13; BMP-14), sonic hedgehog, growth differentiation factors (e.g., GDF5, GDF6, GDF8), recombinant human growth factors (e.g., MP52, and MP-52 variant rhGDF-5), cartilage-derived morphogenic proteins (CDMP-1; CDMP-2, CDMP-3)); small molecules that affect the upregulation of specific growth factors; tenascin-C; hyaluronic acid; chondroitin sulfate; fibronectin; decorin; thromboelastin; thrombin-derived peptides; heparin-binding domains; heparin; heparan sulfate; DNA fragments and DNA plasmids. In addition, the bioactive agent can be an autologous growth factor that is supplied by platelets in the blood. In this case, the growth factor from platelets will be an undefined cocktail of various growth factors. If other such substances have therapeutic value in the orthopaedic field, it is anticipated that at least some of these substances will have use in the present invention, and such substances should be included in the meaning of “bioactive agent”and “bioactive agents” unless expressly limited otherwise. Preferred examples of bioactive agents include culture media, bone morphogenic proteins, growth factors, growth differentiation factors, recombinant human growth factors, cartilage-derived morphogenic proteins, hydrogels, polymers, autologous, allogenic or xenologous cells such as stem cells, chondrocytes, fibroblast and proteins such as collagen and hyaluronic acid. Bioactive agents can be autologus, allogenic, xenogenic or recombinant.

Bioactive agents which act as osteogenic agents are preferred and include but are not limited to hydroxyapatite, tricalcium phosphate, ceramic glass, amorphous calcium phosphate, porous ceramic particles or powders, demineralized bone particles or powder, transforming growth factors (e.g., TGF-β I-III), growth differentiation factors (e.g., GDF5, GDF6, GDF8), bone morphogenic proteins (BMP-2, BMP-4; BMP-6; BMP-7; BMP-12; BMP-13; BMP-14), recombinant human growth factors (such as MP-52 and its variant rhGDF-5), cartilage-derived morphogenic proteins (CDMP-1; CDMP-2, CDMP-3) and combinations thereof.

The bioactive agents can take the form of immediate release (injection) or delayed release using microspheres, nanospheres or other matrices such as hydrogels for controlled release delivery to encourage disc tissue incorporation and regeneration.

Another aspect of the present invention is the ability of the injectable material to degrade at a rate that is amenable to bone replacement.

Another aspect of the present invention is the use of light to cure the injectable material.

Another aspect of the present invention is the use of porogens that dissolve quickly and form interconnected pores throughout the injectable material. The pores allow cellular infiltration.

Several examples of the soft tissue fixation technique are shown in the attachments.

It should be understood that the foregoing disclosure and description of the present invention are illustrative and explanatory thereof and various changes in the size, shape and materials as well as in the description of the preferred embodiment may be made without departing from the spirit of the invention.

What is claimed is:

1. A method for securing a soft tissue implant into bone comprising the steps of:
a) providing a hole in the bone;
b) inserting an end of the soft tissue implant into the hole; and
c) filling the hole with a curable material.

2. The method of claim 1, wherein the hole comprises an undercut.

3. The method of claim 1, wherein the soft tissue implant is a ligament.

4. The method of claim 1, wherein the soft tissue implant is a material selected from the group consisting of elastomeric polymers and extracellular matrices.

5. The method of claim 4 wherein the elastomeric polymers are selected from the group aliphatic polyesters, poly(amino acids), copoly(ether-esters), polyalkylenes oxalates, polyamides, tyrosine derived polycarbonates, poly(iminocarbonates), polyorthoesters, polyoxaesters, polyamidoesters, polyoxaesters containing amine groups, poly(anhydrides), polyphosphazenes, poly(propylene fumarate), polyurethane, poly(ester urethane), poly(ether urethane), and blends and copolymers thereof.

6. The method of claim 5, wherein the elastomeric polymer is selected form the group consisting of ε-caprolactone, glycolide, lactide, p-dioxanone, trimethylene carbonate and combinations thereof.

7. The method of claim 4, wherein the extracellular matrix is selected form the group consisting of small intestine submucosa, stomach, bladder, alimentary, respiratory, genital submucosa, liver basement membrane and combinations thereof.

8. The method of claim 4, wherein the extracellular matrix is small intestine submucosa.

9. The method of claim 1, wherein the curable material is selected form the group consisting of bone cements, resorbable or non-resorbable polymers, tissue adhesives, and biological adhesives

10. The method of claim 1, wherein the curable material comprises polypropylene fumarate or polymethyl methacrylate (PMMA) combined with a cross-linking agent.

11. The method of claim 1, wherein the curable material comprises a thermoplastic or thermosetting polymer.

12. The method of claim 1, wherein the curable material comprises a polymer of polyamino acid or polyanhydride.

13. The method of claim 12, wherein the curable material further comprises tricalcium phosphate, calcium sulfate or hollow PMMA microspheres.

14. The method of claim 1, wherein the curable material comprises a UV curable polymer.

15. The method of claim 9, wherein the curable material further comprises a bioactive agent which acts as an osteogenic agent and is selected from the group consisting of hydroxyapatite, tricalcium phosphate, ceramic glass, amorphous calcium phosphate, porous ceramic particles or powders, demineralized bone particles or powder, transforming growth factors, growth differentiation factors, bone morphogenic proteins, recombinant human growth factors, cartilage-derived morphogenic proteins and combinations thereof.

16. The method of claim 15, wherein the bioactive agent is BMP-2.

17. The method of claim 15, wherein the bioactive agent is BMP-7

18. The method of claim 15, wherein the bioactive agent is rhGDF-5.

19. The method of claim 15, wherein the bioactive agent is hydroxyapatite.

20. The method of claim 15 wherein, the bioactive agent is tricalcium phosphate.

21. The method of claim 1, wherein step c) further comprises inserting into the hole a coil or spring along with the end of the implant.

22. The method of claim 1, wherein step c) further comprises inserting into the hole a shape memory device along with the end of the implant.
